(19) United States
(12) Patent Application Publication
(10) Pub. No.: US 2013/0045526 A1
(43) Pub. Date: Feb. 21, 2013

(54) Method and device for enhancing degradation of petroleum and its derivatives in an object

(71) Applicant: Chitsan Lin, Kaohsiung City (TW)

(72) Inventor: Chitsan Lin, Kaohsiung City (TW)

(21) Appl. No.: 13/137,416

(22) Filed: Aug. 15, 2011

(51) Int. Cl. C12M 1/00; C12S 99/00

(52) U.S. Cl. 435/262.5; 435/289.1

(57) ABSTRACT

A method for enhancing degradation of petroleum and its derivatives in an object includes placing an object containing petroleum and its derivatives into a compartment of a tank. Oil-eating bacteria is added into or cultivated in the compartment. A suction motor is activated to suck air out of the compartment, such that an air pressure in the compartment is smaller than an air pressure outside of the compartment, forcing the air outside of the compartment to flow into the compartment. A device for enhancing degradation of petroleum and its derivatives in an object includes a tank having a space and an outlet in communication with a bottom of the compartment. The tank includes a top side having an opening in communication with the compartment. A suction motor includes an air inlet and an air outlet. The outlet of the tank is connected to the air inlet of the suction motor.

### BACKGROUND OF THE INVENTION

The present invention relates to a method and a device for degrading petroleum and its derivatives in an object and, more particularly, to a method and a device for enhancing degradation of petroleum and its derivatives in an object.

Petroleum and its derivatives (such as gasoline, diesel, kerosene, and fuels for airplanes, ships, and industrial boilers) obtained by refining petroleum are important resources for modern industries and daily articles. However, petroleum and its derivatives, if not properly used, could contaminate the environment. Particularly, the crops grown in coils contaminated by petroleum and its derivatives will be contaminated too and will significantly affect the health of the consumers.

Scientists have found oil-eating bacteria in microorganisms, such as acinetobacter sp, pseudoxanthomonas sp, bacillus sp, etc. These oil-eating bacteria obtain nutrient required for survival by degrading oils, such as animal oils, plant oils, petroleum and its derivatives, edible oils, etc. Generally, food waste is apt to generate oil-eating bacteria during the composting procedure. Taiwan Patent Publication No. 200904991 filed by Applicant on Feb. 1, 2009 discloses a method for biodegrading oils, wherein the oils are biodegraded by cometabolism during the composting procedure of the food waste. Specifically, the method includes providing a mixture of food waste and compost. The mixture of food waste and compost is mixed with an object containing oils to be degraded, obtaining a cometabolic mixture of food waste and compost. The cometabolic mixture of food waste and compost undergoes the composting procedure until a significant amount of oils is decomposed. By the cometabolism during the composting procedure of the cometabolic mixture of food waste and compost, the number of oil-eating bacteria can be largely increased in the food waste to rapidly decompose the carbohydrates, organic nitrogen, and fats in the cometabolic mixture of food waste and compost while degrading the oils.

However, in addition to oils, the oil-eating bacteria require sufficient oxygen for propagation. The admixture must be turned over while using the composting procedure to handle soil contaminated by petroleum and its derivatives, so that the gaps in the admixture can be in contact with the air to provide the oxygen required for survival of the oil-eating bacteria. In the method for handling soil contaminated by petroleum and its derivatives, since the air can not flow between the gaps in the admixture to provide sufficient oxygen required for survival of the oil-eating bacteria, considerable time is required for turning over the admixture during the composting procedure to force the air to enter the gaps, which is time-consuming and wastes manpower and energy while having difficulties in controlling the aeration of the compost.

Thus, a need exists for a novel method for handling soil contaminated by petroleum and its derivatives.

### BRIEF SUMMARY OF THE INVENTION

A method for enhancing degradation of petroleum and its derivatives in an object according to the present invention includes obtaining an object containing petroleum and its derivatives and placing the object in a compartment of a tank. Oil-eating bacteria is added into or cultivated in the compartment. A suction motor connected by a pipe to the compartment is activated to suck air out of the compartment, such that an air pressure in the compartment is smaller than an air pressure outside of the compartment, forcing the air outside of the compartment to flow into the compartment via an opening of the tank.

By providing the method for enhancing degradation of petroleum and its derivatives, when the oil-eating bacteria and the object containing petroleum and its derivatives to be degraded are placed in the compartment, the suction motor sucks air out of the compartment, such that an air pressure in the compartment is smaller than an air pressure outside of the compartment, forcing air to flow in the compartment. Thus, the oil-eating bacteria can propagate in an environment rich with oxygen and petroleum and its derivatives. A better degradation effect of petroleum and its derivatives can be obtained to reduce the content of petroleum and its derivatives in the object, achieving the objective of degradation of petroleum and its derivatives.

A device for enhancing degradation of petroleum and its derivatives in an object according to the present invention includes a tank having a space and an outlet. The outlet is in communication with a bottom of the compartment. The tank includes a top side having an opening in communication with the compartment. A suction motor includes an air inlet and an air outlet. A pipe includes two ends respectively connected to the outlet of the tank and the air inlet of the suction motor.

By providing the device for enhancing degradation of petroleum and its derivatives, when the oil-eating bacteria and the object to be degraded are placed in the compartment, the suction motor continuously sucks air out of the compartment, such that an air pressure in the compartment is smaller than an air pressure outside of the compartment, forcing air to flow in the compartment to provide the oil-eating bacteria with sufficient oxygen for propagation.

The present invention will become clearer in light of the following detailed description of illustrative embodiments of this invention described in connection with the drawings.

### DESCRIPTION OF THE DRAWINGS

FIG. 1 shows a block diagram illustrating a method for enhancing degradation of petroleum and its derivatives in an object according to the present invention.

FIG. 2 shows a diagrammatic view of a device for enhancing degradation of petroleum and its derivatives in an object according to the present invention.

FIG. 3 shows a diagram illustrating the degradation result obtained by using the method and the device for enhancing degradation of petroleum and its derivatives in an object according to the present invention.

### DETAILED DESCRIPTION OF THE INVENTION

Since the oil-eating bacteria need oils (such as animal oils, plant oils, petroleum and its derivatives, edible oils, etc) and degrades the oils to obtain nutrients required for survival, the present invention utilizes oil-eating bacteria to degrade petroleum and its derivatives in an object so as to reduce the content of petroleum and its derivatives in the object. The term “degradation” referred to herein means breaking each molecular link in petroleum and its derivatives to reduce the content of petroleum and its derivatives in the object.

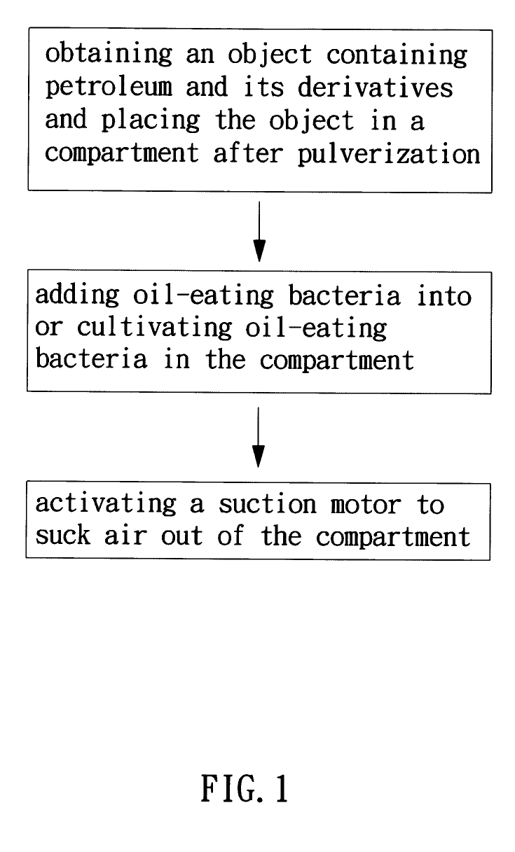

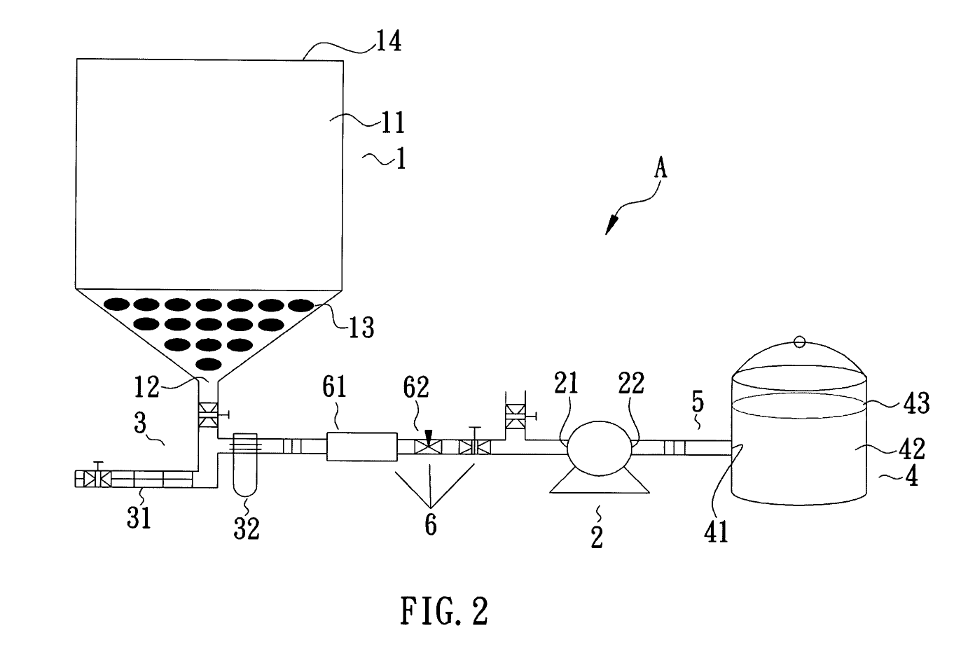

With reference to FIGS. 1 and 2, a method for enhancing degradation of petroleum and its derivatives in an object according to the present invention includes the following steps of: obtaining an object containing petroleum and its derivatives and placing the object in a compartment 11 of a tank 1; adding oil-eating bacteria into or cultivating oil-eating bacteria in the compartment 11; and activating a suction motor 2 connected by a first pipe 3 to the compartment 11 to suck air out of the compartment 11, such that an air pressure in the compartment 11 is smaller than an air pressure outside of the compartment 11, forcing the air outside of the compartment 11 to flow into the compartment 11 via an opening 14 of the tank 1.

A device A for enhancing degradation of petroleum and its derivatives in an object according to the present invention includes a tank 1, a suction motor 2, and a first tube 3. The tank 1 can be of any shape. Preferably, the tank 1 includes circular or rectangular cross sections. The tank 1 includes a compartment 11 and an outlet 12. An opening 14 is defined in a top side of the tank 1 and in communication with a bottom of the compartment 11. Preferably, a filtering material 13 is mounted in the bottom of the compartment 11. The filtering material 13 can be, but not limited to, active charcoal.

The suction motor 2 includes an air inlet 21 and an air outlet 22. The first pipe 3 includes two ends respectively connected to the air inlet 21 and the outlet 12.

The device A can be used to degrade an object containing petroleum and its derivatives (such as gasoline, diesel, kerosene, and fuels for airplanes, ships, and industrial boilers) obtained by refining petroleum. The object to be degraded can be, but not limited to, soil.

The object is pulverized to have a diameter preferably smaller than 1 cm, such that the object can have more gaps for survival of the oil-eating bacteria. The object is then placed in the compartment 11. The oil-eating bacteria is added into or cultivated in the compartment 11. The oil-eating bacteria can be, but not limited to, acinetobacter sp, pseudoxanthomonas sp, bacillus sp, etc. In the preferred embodiment, the oil-eating bacteria are cultivated by food waste that is pulverized before cultivation.

To provide the oil-eating bacteria with a better living environment for propagation, nutrient can be added into the compartment 11. The nutrient includes hydrocarbons containing nitrogen and phosphorus. Preferably, the nutrient includes one of NH₄NO₃, Na₂HPO₄, and KH₂PO₄, or any nutrient containing nitrogen and phosphorus. The temperature of the compartment 11 is kept in a range between 65° C. and 80° C.

The suction motor 2 is activated to suck the air out of the compartment 11, such that the air pressure in the compartment 11 is smaller than the air pressure outside of the compartment 11. The air outside the compartment 11 flows into the compartment 11 via the opening 14 to supply air into the compartment 11.

Due to flow of air in the compartment 11, the oil-eating bacteria are provided with sufficient oxygen for propagation and degrade the petroleum and its derivatives contained in the object, reducing the content of petroleum and its derivatives in the object. When the object contaminated by petroleum and its derivatives is soil, by using the method and device for enhancing degradation of petroleum and its derivatives according to the present invention, the soil can rapidly be restored to the uncontaminated state so that people can use the soil to grow crops.

Generally, food waste is rich with (such as nitrogen and phosphorus) required by oil-eating bacteria. During the composting procedure of food waste, the temperature of the food waste can be increased up to above 65° C., which is suitable for propagation of the thermophilic oil-eating bacteria. Thus, in the method for enhancing degradation of petroleum and its derivatives, cultivation of the oil-eating bacteria includes mixing the object with the food waste to obtain an admixture, and the admixture is then placed into the compartment 11 to proceed with the composting procedure for propagating the oil-eating bacteria that degrade petroleum and its derivatives in the object to be degraded.

Since filtering material 13 is provided in the bottom of the compartment 11 and includes a plurality of gaps, the object to be degraded is less likely to enter the first pipe 3 while the suction motor 2 sucks air out of the compartment 11 and the gaps of the object. Blockage of the first pipe 3 is, thus, avoided.

In the method for enhancing degradation of petroleum and its derivatives by using food waste to proceed with composting, ferment materials, such as wood dust, hay, sugarcane waste, straws, rice husks, leaves, and waste of mushroom-breeding material, can be added into the food waste to provide the oil-eating bacteria with the carbon source required for survival, to increase the carbon nitrogen ratio, and to adjust the water content of the food waste, providing the oil-eating bacteria with a better growing environment. Thus, the degradation effect of petroleum and its derivatives can be enhanced.

In an embodiment, the device A includes an air filter 4 having a filter inlet 41 and a filtering space 42. The filter inlet 41 is in communication with the filtering space 42. The filtering space 42 receives a deodorizing element 43. When air enters the filtering space 42, the smell in the air can be filtered by the deodorizing element 43. The device A can further include a second pipe 5 having two ends respectively connected to the filter inlet 41 and the air outlet 22.

In the method for enhancing degradation of petroleum and its derivatives by using food waste to cultivate the oil-eating bacteria, since the air in the compartment 11 and the gaps of the admixture will have a smell (the air in the compartment 11 and the gaps of the admixture and having a smell will be referred to as “gas” hereinafter), when the suction motor 2 is sucking away the gas in the compartment 11 and the gaps of the admixture during the composting procedure, the gas will enter the filtering space 42 via the second pipe 5. The smell of the gas will be removed by the deodorizing element 43. Preferably, the deodorizing element 43 is mature compost (i.e., organisms obtained after the composting procedure). Microorganisms contained in the mature compost can remove the smell through decomposition and metabolism.

In another embodiment of the device A, the first pipe 3 further includes a flow control device 6 including a flowmeter 61 and a flow control valve 62. The flowmeter 61 detects the speed of the air in the first pipe 3. The speed of the air in the first pipe 3 can be controlled by the flow control valve 62. The flow control valve 62 can be, but not limited to, a ball valve or a needle valve.

When the speed of the air in the first pipe 3 is larger than a predetermined value, the suction motor 2 is controlled to reduce the air sucking speed, preventing the admixture from entering the first pipe 3 due to the fast moving air while the suction motor 2 is sucking away the air in the compartment 11 and the gaps of the admixture. Blockage of the first pipe 3 is, thus, avoided. Furthermore, the speed of the air in the first pipe 3 can be precisely controlled by using the flow control valve 62.

In a further embodiment of the device A, a waste water pipe 31 extends from the first pipe 3, and a water collecting bottle 32 is provided on the first pipe 3. The waste water pipe 31 is located between the water collecting bottle 32 and the outlet 12.

Since waste water is generated during the composting procedure of the admixture, the waste water will be sucked into the first pipe 3 while the suction motor 2 sucks air out of the compartment 11 and the gaps of the admixture. The waste water pipe 31 proceeds with a first stage of removal of waste water. The remaining waste water is collected in the water colleting bottle 32, which is a second stage of removal of waste water. Thus, the waste water will not enter the suction motor 2, avoiding damage to the suction motor 2.

### Example

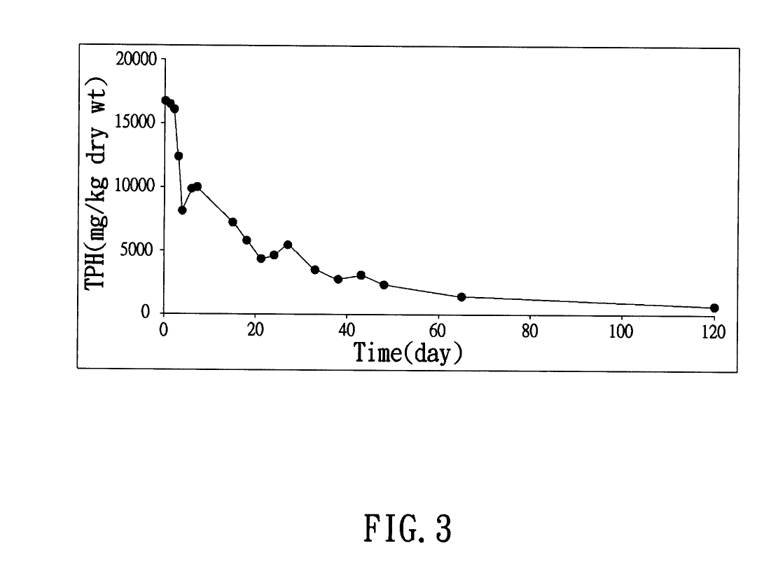

650 kg of food waste was pulverized and then added with 200 kg of wood dust, 150 kg of mature compost, and 7 kg of fuel oil to obtain an admixture having a TPH concentration of 16725 mg/kg dry weight. The admixture was degraded by the method and device for enhancing degradation of petroleum and its derivatives according to the present invention. FIG. 3 shows the degradation result, proving the feasibility and improvement of the present invention. The term “TPH” (total petroleum hydrocarbon) in FIG. 3 means the total amount of petroleum and its derivatives in the admixture.

Measurement of the TPH was conducted by a gas chromatography that can measure compounds having a carbon number between six and forty. In the beginning, the TPH in the admixture was 16725 mg/kg. On the 18^{th} day, the TPH in the admixture dropped to 5796 mg/kg. On the 65^{th} day, the TPH in the admixture dropped to 1400 mg/kg. The degradation rate was about 65% on the 18^{th} day and 92% on the 65^{th} day which are superior than the general biological treatment efficiency for the treatment fuel oils.

Although specific embodiments have been illustrated and described, numerous modifications and variations are still possible without departing from the essence of the invention. The scope of the invention is limited by the accompanying claims.

1. A method for enhancing degradation of petroleum and its derivatives in an object comprising the following steps of:
obtaining an object containing petroleum and its derivatives and placing the object in a compartment of a tank;
adding oil-eating bacteria into or cultivating oil-eating bacteria in the compartment; and
activating a suction motor connected by a pipe to the compartment to suck air out of the compartment, such that an air pressure in the compartment is smaller than an air pressure outside of the compartment, forcing air outside of the compartment to flow into the compartment via an opening of the tank.

2. The method for enhancing degradation of petroleum and its derivatives in an object as claimed in claim 1, wherein the oil-eating bacteria is selected from a group consisting of acinetobacter sp, pseudoxanthomonas sp, and bacillus sp and combinations thereof.

3. The method for enhancing degradation of petroleum and its derivatives in an object as claimed in claim 2, with the method further including adding nutrient into the compartment, with the nutrient being one of NH₄NO₃, Na₂HPO₄, and KH₂PO₄, with a temperature of the compartment being kept in a range between 65° C. and 80° C.

4. The method for enhancing degradation of petroleum and its derivatives in an object as claimed in claim 1, with the method further including adding food waste into the compartment to cultivate the oil-eating bacteria.

5. The method for enhancing degradation of petroleum and its derivatives in an object as claimed in claim 1, wherein the object is pulverized before the object is placed into the compartment.

6. A device for enhancing degradation of petroleum and its derivatives in an object comprising:
a tank including a space and an outlet, with the outlet being in communication with a bottom of the compartment, with the tank including a top side having an opening in communication with the compartment;
a suction motor including an air inlet and an air outlet; and
a first pipe including two ends respectively connected to the outlet of the tank and the air inlet of the suction motor.

7. The device for enhancing degradation of petroleum and its derivatives in an object as claimed in claim 6, further comprising: a waste water pipe extending from the first pipe; and a water collecting bottle mounted on the first pipe, with the waste water pipe located between the water collecting bottle and the outlet of the tank.

8. The device for enhancing degradation of petroleum and its derivatives in an object as claimed in claim 6, further comprising: a flow control device mounted on the first pipe, with the flow control device including a flowmeter and a flow control valve.

9. The device for enhancing degradation of petroleum and its derivatives in an object as claimed in claim 6, further comprising: an air filter including a filter inlet and a filtering space in communication with the filter inlet, with the filtering space receiving a deodorizing element; and a second pipe having two ends respectively connected to the filter inlet and the air outlet.

10. The device for enhancing degradation of petroleum and its derivatives in an object as claimed in claim 6, further comprising: a filtering material mounted in the bottom of the compartment.
